(19) United States
(12) Patent Application Publication
(10) Pub. No.: US 2026/0009122 A1
(43) Pub. Date: Jan. 8, 2026

(54) COMPOUNDS COMPRISING SULFUR AND (METH)ACRYLATE MOIETIES SUITABLE FOR BONDING ORGANIC POLYMERS TO METAL

(71) Applicant: 3M INNOVATIVE PROPERTIES COMPANY, St. Paul, MN (US)

(72) Inventors: Thomas P. Klun, Lakeland, MN (US); Henrik B. van Lengerich, Woodbury, MN (US); Christopher S. Lyons, St. Paul, MN (US); John L. Battiste, Northfield, MN (US)

(21) Appl. No.: 19/118,577

(22) PCT Filed: Nov. 15, 2023
(86) PCT No.: PCT/IB2023/061549

§ 371 Date: Apr. 4, 2025

Related U.S. Application Data
(60) Provisional application No. 63/432,869, filed on Dec. 15, 2022.

(51) Int. Cl. C23C 14/20; C07C 323/52; C08G 75/045; C23C 14/34; C23C 16/24; C23C 16/505

(52) U.S. Cl. CPC C23C 14/20; C07C 323/52; C08G 75/045; C23C 14/34; C23C 16/24; C23C 16/505

(57) ABSTRACT

An article is described comprising an organic polymer layer comprising a compound comprising at least one sulfur moiety and at least one (meth)acrylate moiety; wherein a surface of the organic polymer layer is bonded to a (e.g. thin layer of) metal, such as gold or silver. Also described are various compounds comprising at least one sulfur moiety and at least one (meth)acrylate moiety, methods of making such compounds, and polymerizable resins comprising such compounds.

### SUMMARY

In one embodiment, an article is described comprising an organic polymer layer comprising a compound comprising at least one sulfur moiety and at least one (meth)acrylate moiety; wherein a surface of the organic polymer layer is bonded to metal. In some embodiments, the metal comprises gold or silver. In some embodiments, the metal has a thickness of no greater than 5 microns, 1 micron, 500 nm, 250 nm, 100 nm, or 50 nm.

The compound comprising at least one sulfur moiety and at least one (meth)acrylate moiety may comprise one or more novel compounds.

In other embodiments, various compounds comprising at least one sulfur moiety and at least one (meth)acrylate moiety are described.

In one embodiment, the compound is the Michael addition reaction product of a multithiol compound; one or more diacrylate compounds; and a base.

In one embodiment, the compound has Formula I:

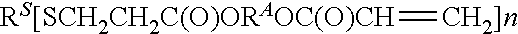

wherein
R^{S} is the residue of a multithiol,
R^{A} is independently a residue of a diacrylate, and
n is 2 to 6. (e.g. the number of thiol groups of the multithiol)

In another embodiment, the compound is the reaction product of a multithiol compound; one or more isocyanato(meth)acrylate compounds; and a base. The reaction product comprises compounds with at least one isocyanato(meth)acrylate group and one or more thiol groups.

In one embodiment, the compound has Formula II:

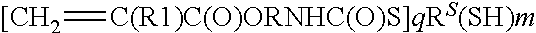

wherein

### R1 is H or CH₃,

R is a linear or branched alkylene of 2 to 10 carbons, optionally substituted with O,
R^{S} is the residue of a multithiol,
q is 1 to 5, and
m is 1 to 5, (e.g. the number of unreacted thiol groups of the multithiol)
with the proviso that the sum of q+m ranges from 2 to 6. (e.g. the number of thiol groups of the multithiol).

In another embodiment, the compound is the Michael addition reaction product of the compound comprising at least one isocyanato(meth)acrylate group and one or more thiol groups (as just described) and a diacrylate compound.

In one embodiment, the compound has Formula III:

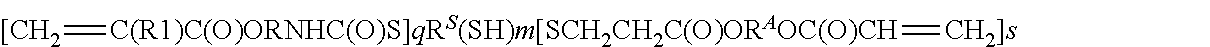

wherein

### R1 is H or CH₃,

R is a linear or branched alkylene of 2 to 10 carbons, optionally substituted with O,
R^{S} is the residue of a multithiol,
q is 1 to 5,
m is 0 to 4, (e.g. the number of unreacted thiol groups of the multithiol) sis 1 to 5,
with the proviso that the sum of q+m+s ranges from 2 to 6. (e.g. the number of thiol groups of the multithiol)

In some embodiments, m is at least 1.

Also described is a polymerizable resin composition comprising one or more (meth)acrylate monomers; and a compound comprising at least one sulfur moiety and at least one (meth)acrylate moiety, as described herein.

Also described are methods of making compound comprising at least one sulfur moiety and at least one (meth)acrylate moiety. The compounds may be prepared in an organic solvent or solventless methods can be employed, such as forming the compound in a polymerizable resin.

### BRIEF DESCRIPTION OF THE DRAWINGS

FIG. 1 is a schematic diagram of a two-layer article;

FIG. 2 is a schematic diagram of a three-layer article;

FIG. 3 is a schematic diagram of an assembly of layers utilized to evaluate the bond strength of a metal layer bond to a cured resin.

### DETAILED DESCRIPTION

Presently described are compounds comprising sulfur and (meth)acrylate moieties. The compounds are useful as coupling agent for bonding an organic polymer layer, such as a cured organic resin to metal.

The compounds comprising sulfur and acrylate moieties are prepared by reaction of a multithiol compound. In some embodiments, the multithiol compounds and the reaction product thereof are aliphatic. In other embodiments, the multithiol compounds and the reaction product are aromatic multithiols.

Some examples of multithiols include aliphatic thiols such as methanedithiol, propanedithiol, cyclohexanedithiol, 2-mercaptoethyl-2,3-dimercaptosuceinate, 2,3-dimercapto-1-propanol(2-mercaptoacetate), diethylene glycol bis(2-mercaptoacetate), 1,2-dimercaptopropyl methyl ether, bis(2-mercaptoethyl)ether, trimethylolpropane tris(thioglycolate), pentaerythritol tetra(mercaptopropionate), pentaerythritol tetra(thioglycolate), ethylene glycol dithioglycolate, trimethylolpropane tris(beta-thiopropionate), tris-mercaptan derivative of tri-glycidyl ether of propoxylated alkane, and dipentacrythritol poly(beta-thiopropionate); halogen-substituted derivatives of the aliphatic thiols; aromatic thiols such as di-, tris- or tetra-mercaptobenzene, bis-, tris- or tetra-(mercaptoalkyl) benzene, dimercaptobiphenyl, toluenedithiol and naphthalenedithiol; halogen-substituted derivatives of the aromatic thiols; heterocyclic ring-containing thiols such as amino-4,6-dithiol-sym-triazine, alkoxy-4,6-dithiol-sym-triazine, aryloxy-4,6-dithiol-sym-triazine and 1,3,5-tris(3-mercaptopropyl) isocyanurate; halogen-substituted derivatives of the heterocyclic ring-containing thiols; thiol compounds having at least two mercapto groups and containing sulfur atoms in addition to the mercapto groups such as bis-, tris- or tetra(mercaptoalkylthio)benzene, bis-, tris- or tetra(mercaptoalkylthio)alkane, bis(mercaptoalkyl) disulfide, hydroxyalkylsulfidebis(mereaptopropionate), hydroxyalkylsulfidebis(mercapto acetate), mercaptoethyl ether bis(mercaptopropionate), 1,4-dithian-2,5-diolbis(mercaptoacetate), thiodiglycolic acid bis(mercaptoalkyl ester), thiodipropionie acid bis(2-mercaptoalkyl ester), 4,4-thiobutyric acid bis(2-mercaptoalkyl ester), 3,4-thiophenedithiol, bismuththiol and 2,5-dimercapto-1,3,4-thiadiazol.

Various multithiol compounds are commercially available. Some illustrative multithiol compounds are as follows:

| | |
|---|---|
| DMDO | HSCH₂CH₂OCH₂CH₂OCH₂CH₂SH, CAS number 14970-87-7, obtained |
| | as “DMDO” from Arkema, King of Prussia, PA. |
| GDMP | 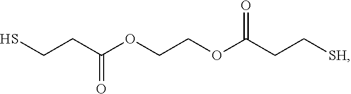 [SMILES: O=C(CCS)OCCOC(=O)CCS] |
| | CAS number 22504-50-3, thiol |
| | equivalent weight 123.5, obtained from Bruno Bock, Marschacht, |
| | Germany |
| PEI | 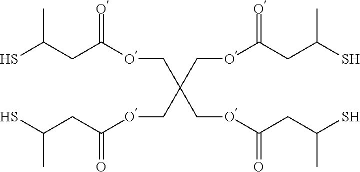 |
| | CAS numbers 31775-89-0, and |
| | 1027326-93-7, thiol equivalent weight 135, obtained as “KarenzMT ™ |
| | PEI” from Showa Denko, Tokyo, Japan. |
| PETMP | 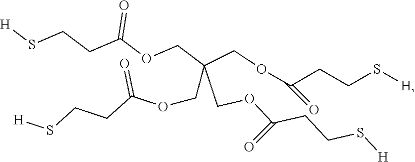 |
| | CAS 7575-23-7, Thiol |
| | equivalent weight 126.5, obtained from Evans Chemetics LP, Waterloo, |
| | New York. |
| TEMPIC | 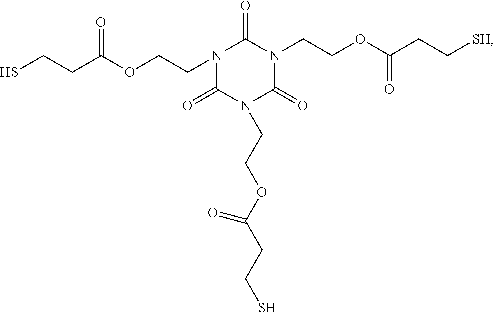 [SMILES: O=C(CCS)OCCn1c(=O)n(CCOC(=O)CCS)c(=O)n(CCOC(=O)CCS)c1=O] |
| | CAS number 928339-75-7, thiol |
| | equivalent weight 182, obtained from Bruno Bock. |
| TMMP | 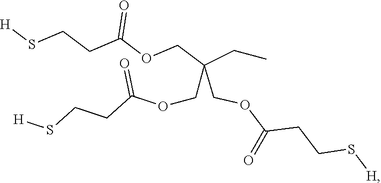 |
| | CAS number 33007-83-9, |
| | thiol equivalent weight 138, obtained as “TMMP-LV” from Kowa, New |
| | York, New York. |

After reaction with an acrylate or (meth)acrylate group in the presence of a base, a residue of a multithiol remains. The residue of a multithiol is the organic group between the sulfur atoms of the multithiol. For example, when the multithiol has the following structure:

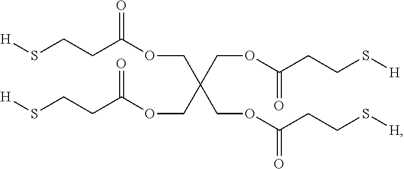

the residue of the multithiol is

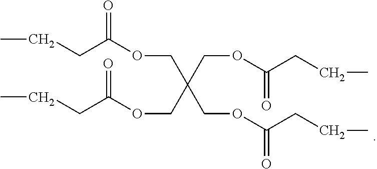 [SMILES: CCCC(=O)OCC(COC(=O)CCC)(COC(=O)CCC)COC(=O)CCC]

The residue of a multithiol is a linear or branched organic group. When the multithiol has three or more thiol groups, there is typically a branch for each thiol group, as depicted above. The organic group may comprise various organic moieties including alkylene, arylene, alkarylene, aralkylene; moieties with oxygen atoms such as ether or ester; and moieties with nitrogen atoms such as isocyanurate. The organic group may comprise cycloaliphatic or heterocycloaliphatic moieties. In some embodiments, the organic group comprises combinations of such moieties.

In some embodiments, the multithiol compounds comprise ester moieties. In some embodiments, the ratio of ester moieties to thiol moieties is 1:1. Thus, such compounds may be characterized as thioesters. In some embodiments, the multithiol compounds further comprise a heterocyclic group, such as an isocyanurate group.

In some embodiments, the multithiol compounds comprise at least 3 or 4 thiol groups. In some embodiments, higher number of thiol group is amenable to forming the desired reaction product in less time. For example, PETMP (depicted below) can form the desired reaction product within 2.5 hours. The number of thiol groups is typically no greater than 6, 5, or 4.

The multithiol compounds (and residue thereof) typically have a molecular weight no greater than about 1200 or 1000 g/mole. Further the thiol equivalent weight, or in other words the molecular weight divided by the number of thiol groups, is typically at least about 100 or 125 grams/thiol group. In some embodiments, the thiol equivalent weight is no greater than 250, 200, 20 or 150 grams/thiol group.

The methods of making the multithiol compounds described herein generally comprise reacting a multithiol with one or more diacrylate monomers or an isocyanato(meth)acrylate compounds in the presence of a base. In some embodiments, the method of making the multithiol compounds comprises reacting a multithiol with an isocyanato(meth)acrylate to form an intermediate and reacting the intermediate with a diacrylate monomers.

Various bases can be utilized. In some embodiments, phosphine bases such as dimethyl phenyl phosphine are utilized. In other embodiments, the base is an amine compound. Amine compound includes primary, secondary, and tertiary amines. Illustrative amine compounds include alkyl amines (e.g. triethylamine, diisopropylethylamine); alkyl amino acrylates (e.g. 2-(dimethylamino)ethyl acrylate), and hydroxyl functional amines (e.g. N,N-dimethylaminoethanol). In some embodiments, phosphine bases such as dimethyl phenyl phosphine. The amount of base is typically at least 0.025, 0.050, 0.10, 0.25, 0.50 parts by weight compared to 100 total parts of diacrylate and multithiol. In some embodiments, the amount of base is typically no greater than 2.5, 2.0, 1.5, 1.0, or 0.50 parts by weight compared to 100 total parts of diacrylate and multithiol. Lower concentrations of base may require longer reaction times.

Tertiary (e.g. alkyl) amines, especially diisopropylethylamine, can be favored for shorter reaction times. Other tertiary amines include methyldiethanolamine, triethanolamine, diethylaminopropylamine, benzyldimethyl amine, m-xylylenedi(dimethylamine), N,N′-dimethylpiperazine, N-methylpyrrolidine, N-methylhydroxypiperidine, N,N,N′,N′-tetramethyldiaminoethane. N,N,N′,N′,N′-pentamethyldiethylenetriamine, tributyl amine, trimethyl amine, diethyldecyl amine, triethylene diamine, N-methyl morpholine, N,N,N′N′-tetramethyl propane diamine, N-methyl piperidine, N,N′-dimethyl-1,3-(4-piperidino) propane, pyrridine; 1,8-diazobicyclo[5.4.0]undec-7-ene, 1,8-diazabicyclo[2.2.2]octane, 4-dimethylaminopyrridine, and 4-(N-pyrrolidino)pyrridine, triethyl amine and 2,4,6-tris(dimethylaminomethyl)phenol.

Synthetic reactions can be carried out in organic solvent or in the absence of solvent. Organic solvents include ketones such as acetone, methyl ethyl ketone (MEK), methyl isobutyl ketone, methyl amyl ketone and N-methyl pyrrolidone (NMP); ethers such as tetrahydrofuran, 2-methyl tetrahydrofuran and methyl tetrahydrofurfuryl ether: esters such as methyl acetate, ethyl acetate and butyl acetate; cyclic esters such as delta-valerolactone and gamma-valerolactone.

Examples of diacrylate monomers include 1,3-butylene glycol diacrylate, 1,4-butanediol diacrylate, 1,6-hexanediol diacrylate, ethylene glycol diacrylate, alkoxylated aliphatic diacrylate, alkoxylated cyclohexanedimethanol diacrylate, alkoxylated hexanediol diacrylate, alkoxylated neopentyl glycol diacrylate, caprolactone modified neopentyl glycol hydroxypivalate diacrylate, caprolactone modified neopentyl glycol hydroxypivalate diacrylate, cyclohexanedimethanol diacrylate, diethylene glycol diacrylate, dipropylene glycol diacrylate, ethoxylated bisphenol A diacrylate, hydroxypivalaldehyde modified trimethylolpropane diacrylate, neopentyl glycol diacrylate, polyethylene glycol diacrylate, propoxylated neopentyl glycol diacrylate, tetraethylene glycol diacrylate, tricyclodecanedimethanol diacrylate, triethylene glycol diacrylate, and tripropylene glycol diacrylate.

In some embodiments, the diacrylate monomers have a molecular weight no greater than 500, 400, or 300 g/mole. In other embodiments, the diacrylate monomers have a higher molecular weight, of greater than 500, 750, 1000, 1250, or 1500 g/mole. In some embodiments, the molecular weight is no greater than 5000, 4500, 4000, 3500, 3000, 2500, or 2000 g/mole. Such higher molecular weight monomers may be characterized as oligomers.

Oligomeric diacrylate monomers include, for example, urethane acrylates, polyester acrylates, and epoxy acrylates can also be employed. In some embodiments, the oligomer may be derived from a polycaprolactone diol and H12MDI (dicyclohexyl methane diisocyanate, sold as Desmodur W by Covestro).

In some embodiments, the diacrylate oligomer comprises an aliphatic urethane acrylate oligomer having a tensile strength of less than 10,000 kPa; an elongation of 30-50%, a modulus ranging from 50,000 to 10,000 kPa; and a glass transition temperature ranging from 25 to 50° C.

The reaction mixture may optionally comprise multi(meth)acrylate having greater than two (meth)acrylate groups. Examples include tri(meth)acrylate containing monomers such as glycerol triacrylate, trimethylolpropane triacrylate, ethoxylated triacrylates (e.g., ethoxylated trimethylolpropane triacrylate), propoxylated triacrylates (e.g., propoxylated glyceryl triacrylate, propoxylated trimethylolpropane triacrylate), trimethylolpropane triacrylate, tris(2-hydroxyethyl)isocyanurate triacrylate; and higher functionality (meth)acrylate containing monomers such as ditrimethylolpropane tetraacrylate, dipentaerythritol pentaacrylate, pentaerythritol tetraacrylate, ethoxylated pentaerythritol tetraacrylate, and caprolactone modified dipentaerythritol hexaacrylate.

In one embodiment, a method of making a compound (e.g. via Michael addition) is described comprising reacting a multithiol compound with one or more diacrylate compounds in the presence of a base. In this embodiment, the compound is the Michael addition reaction product of a multithiol compound; one or more diacrylate compounds; and a base.

In this embodiment, such compound may be formed in a substantial excess of (meth)acrylate monomers including diacrylate monomers. In this embodiment, the compound can be formed in a polymerizable (meth)acrylate resin composition in contrast to being prepared separately and added to a polymerizable (meth)acrylate resin composition. It is appreciated that the polymerizable (meth)acrylate resin composition may comprise methacrylate monomers, even though such compounds do not react in a base catalyzed Michael reaction with multithiol compounds.

In this embodiment, the amount of diacrylate acrylate monomers(s) is at least 80, 90, 95, 96, 97, 98, or 99 or 99.9 wt. % based on the total wt. % solids of the reaction mixture (i.e. excluding organic solvent that may be present. The amount of diacrylate monomers can be less than 80 wt. % (e.g. at least 50, 60, or 70 wt. %) if other (meth)acrylate monomers are present.

In this embodiment, the amount of multithiol is typically at least 0.1, 0.5, 1, 2, 3, 4, 5, 6, 7, 8, 9 or 10 wt. % based on the total wt. % solids of the reaction mixture (i.e. excluding organic solvent and base that may be present.) The amount of multithiol is typically less than 20 wt. %. In some embodiments, the amount of multithiol is less than 15, 14, 13, 12, 11, or 10 wt. %. Likewise, the polymerizable resin and the organic polymer layer that comprise the cured multithiol acrylate compound may comprise less than 15, 14, 13, 12, 11, or 10 wt. % of moieties (residues) of a multithiol.

In this embodiment, the method and compound may comprise an equivalent ratio of acrylate groups to thiol groups in a range from 33:1 to 3:1. As demonstrated in the forthcoming examples, when the ratio is too low, the adhesion can be poor resulting in the assembly of layers peeling at interface D (as further described in the forthcoming examples). In some embodiments, the equivalent ratio of acrylate groups to thiol groups is at least 4:1, 5:1, 6:1, 7:1, 8:1, 9:1 or 10:1. In other embodiments, the equivalent ratio of acrylate groups to thiol groups is at least 15:1, 20:1, 25:1, or 30:1. In other embodiments, the equivalent ratio of acrylate groups to thiol groups is less than 30:1, 25:1, 20:1, 15:1 or 10:1. Lower ratios can occur when the compound is synthesized in a smaller quantity of diacrylate compounds that was later added to the remainder of the polymerizable resin (that may comprise other (meth)acrylate monomer(s)).

A representative reaction scheme is as follows:

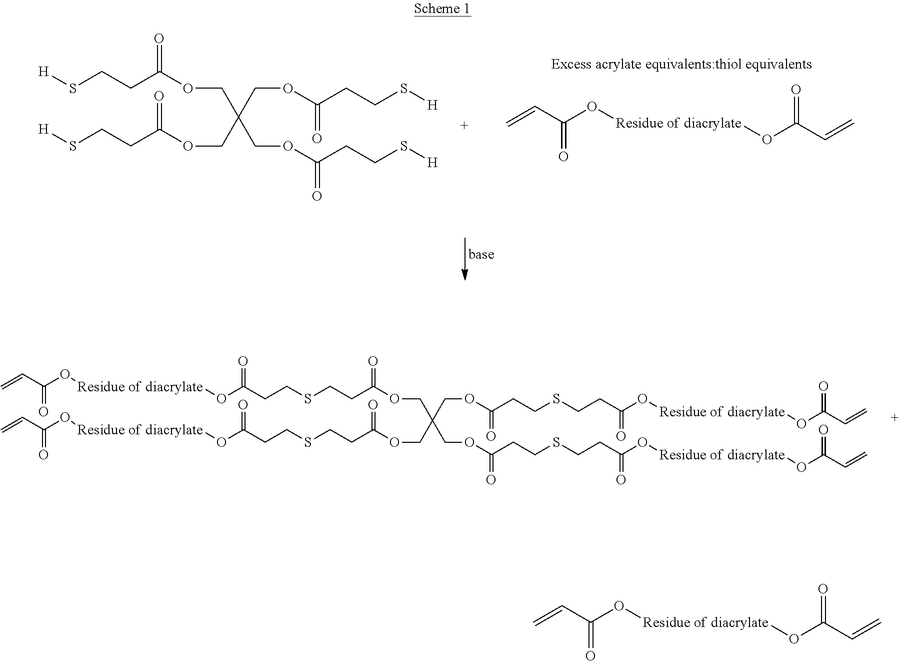

The “residue of diacrylate” is the organic group that is between the oxygen atoms of the acrylate groups. For example, the residue of hexane diol diacrylate is —CH₂CH₂CH₂CH₂CH₂CH₂—. In other embodiments, the diacrylate has the following structure:

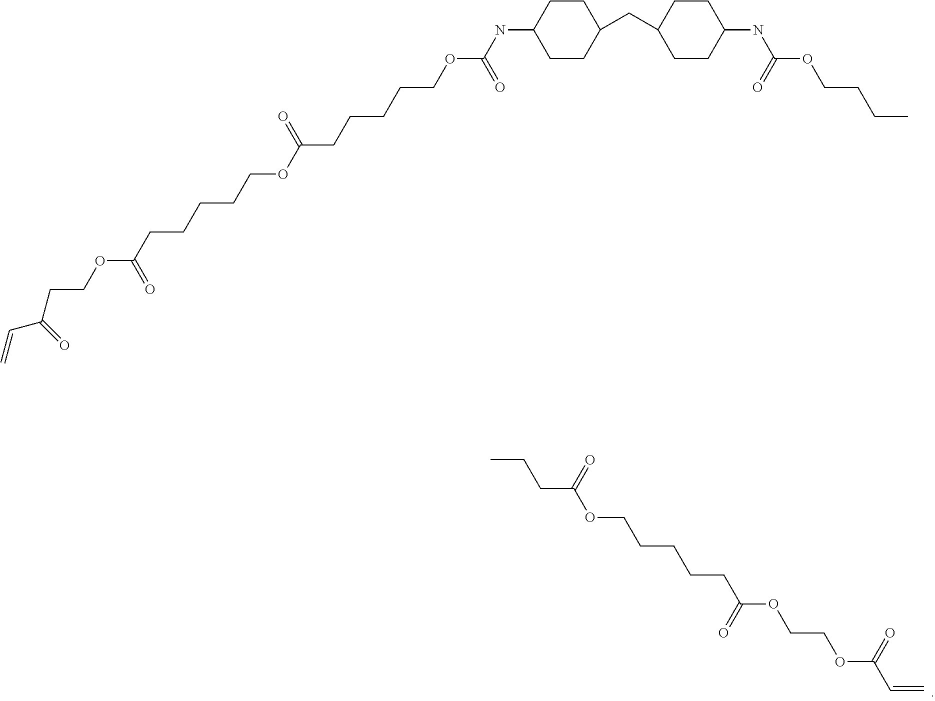

Further, a portion of the “residues of diacrylate” may be a residue of a multiacrylate having three of more acrylate groups, as previously described.

The residue of a multiacrylate (e.g. diacrylate) is a linear or branched organic group. The organic group may comprise various organic moieties including alkylene, arylene, alkarylene, aralkylene; moieties with oxygen atoms such as ether or ester; and moieties with nitrogen atoms such as urethane or isocyanurate. The organic group may comprise cycloaliphatic or heterocycloaliphatic moieties. In some embodiments, the organic group comprises combinations of such moieties.

When a mixture of diacrylates are reacted with a multithiol, the reaction product typically comprises residues of each multiacrylates at about the same equivalent weight fraction as present in the initial reaction mixture. For example, when 100 g the multiacrylate contains 75 parts weight parts (75 g) (Photomer 6210) aliphatic urethane and 25 parts (25 g) HDDA. The acrylate equivalent weight of Photomer 6210 is 475.2 g/eq, and 75 g of this material contains 75/475.2=0.1578 eq acrylate. Similarly, the acrylate equivalent weight of HDDA is 226/2=113 g/eq, and 25 g of this material contains 25/113=0.221 eq acrylate. The total number of acrylate equivalents of this 100 g sample is 0.1578+0.221=0.3788. On a percentage basis the amount of acrylate groups present are (0.1578/0.3788)*100=41.66% from Photomer 6210 and 100−41.66=58.343% from HDDA. A typical formulation might contain a 95:5 weight ratio of this acrylate mixture: PETMP (thiol eq weight 126.5). Assuming there is no photoinitiator or base in the 100 g of acrylate mixture for simplicity of calculation, a 95:5 acrylate mixture would proportionally have 5.26 g of PETMP which is 5.26/126.5=0.041 eq of thiol groups. The adduct should then have statistically about 41.66% Photomer 6210 diacrylate residues, and about 58.34% HDDA residues, assuming perfect capping of the multithiol with the diacrylates and equal reactivity of the acrylate materials. The capped material consumes 2*0.041=0.082 eq of the acrylate groups, since the Photomer 6210 and the HDDA are both diacrylates. Thus 41.66% of the 0.082 acrylates equivalent of Photomer 6210 or 0.03416 acrylate equivalents, or 0.017 mol*950.4=16.23 g (due to the fact that the Photomer 6210 is difunctional) are incorporated into the thiol diacrylate adduct. This leaves 0.1578−0.03416=0.12364. acrylate equivalents of Photomer 6210, or 0.06182 moles of Photomer 6210 or 0.06182*950.4-58.75 g of unmodified Photomer in the mixture. For mass balance the amount of Photomer 6210 in the mixture is then 16.23+58.75=74.98 g or 75 g after rounding for using truncated numbers. Similarly, 58.34% of the 0.082 acrylates equivalent of HDDA or 00.04784 acrylate equivalents are incorporated into the thiol diacrylate adduct or 0.02392 mol*226=5.406 g. This leaves 0.221−0.04784=0.1732 acrylate equivalents of HDDA, or 0.08658 moles of HDDA weighing 0.08658*226=19.567 g of unmodified HDDA in the mixture. The mass balance for the HDDA is then 5.406+19.567=24.97 g or 25 g after rounding for using truncated numbers.

It is appreciated that some amount of individual compounds comprise residues of the same diacrylate (e.g. HDDA). It is also appreciated that some individual compounds may comprise unreacted thiol groups.

In one embodiment, the compound has Formula I:

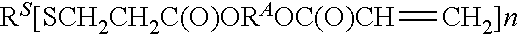

wherein
R^{S} is the residue of a multithiol,
R^{A} is independently a residue of a diacrylate, and
n is 2 to 6 (e.g. the number of thiol groups of the multithiol).

In some embodiments, n is 3 or 4.

In other embodiments, a method of making a compound is described comprising reacting a multithiol compound with one or more isocyanato(meth)acrylate compounds in the presence of a base. The multithiol compound and base can be the same as previously described. The reactions can be carried out in organic solvent or in the absence of solvent as previously described.

The (meth)acrylate compound can be aliphatic or aromatic. Some representative compounds include isocyanatoethyl methacrylate, available under the trade designation “KARENZ MOI”, isocyanatoethoxyethyl methacrylate, available under the trade designation “KARENZ MOI-EG”, isocyanatoethyl acrylate, available under the trade designation “KARENZ AOI”, and 1, 1-(bisacryloyloxymethyl) ethyl isocyanate, available under the trade designation “KARENZ BEI”, which are for instance commercially available from Showa Denko (Tokyo, Japan). Other examples include the (meth)acryloyl group-containing aromatic isocyanates disclosed in U.S. Pat. No. 8,044,235. Examples include the following formulas in which R² is H or methyl.

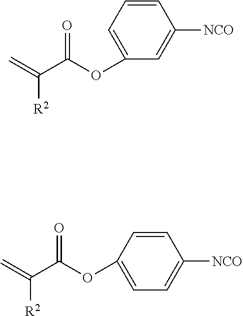

In some embodiments, the amount of reacts is chosen such that there is insufficient isocyanato(meth)acrylate to react with all the thiol groups. In this embodiment, the compound comprises at least one isocyanto(meth)acrylate group and one or more thiol groups.

In this embodiment, the amount of isocyanato(meth)acrylate is of at least 10, 15, 20, 25, 30, 35, 40, 45 wt. % based on the total wt. % solids of the reaction mixture (i.e. excluding organic solvent and base that may be present.) The amount of isocyanato(meth)acrylate is typically no greater than 65, 60, 55, or 50 wt. %

In this embodiment, the amount of multithiol is typically at least 30, 35, 40, 45, 50, 60, 65 wt. % based on the total wt. % solids of the reaction mixture (i.e. excluding organic solvent and base that may be present.) The amount of multithiol is typically less than 85, 80, 75, 70 wt. %.

Representative reaction scheme are as follows:

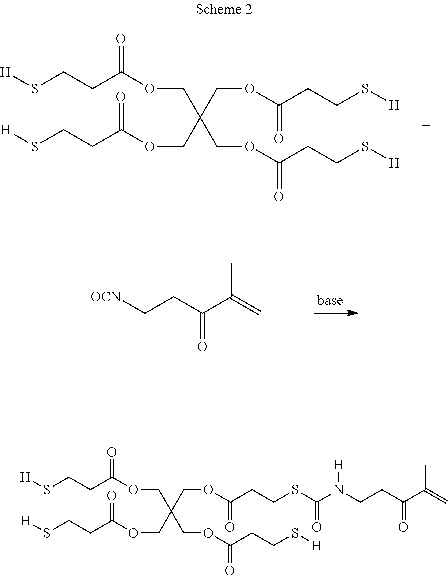

In this reaction scheme, one of the thiol groups has been reacted with the isocyanato(meth)acrylate compound. In other embodiments, more of the thiol groups have been reacted. In typical embodiments, the number of thiol groups that have been reacted with the isocyanato(meth)acrylate compound is greater than one, yet less than all.

In some embodiments, the resulting urethane compound has Formula II:

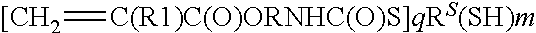

wherein

### R1 is H or CH₃,

R is a linear or branched alkylene of 2 to 10 carbons, optionally substituted with O,
R^{S} is the residue of a multithiol,
q is 1 to 5,
m is 1 to 5, (e.g. the number of unreacted thiol groups of the multithiol),
with the proviso that the sum of q+m ranges from 2 to 6 (e.g. the number of thiol groups of the multithiol).

In some embodiments, q is 2 or 3. In some embodiments, m is 2 or 3. In some embodiments, the sum of q+m is 3 or 4.

In yet other embodiments, a method of making a compound is described comprising (e.g. Michael addition) reacting a multithiol compound comprising at least one isocyanato(meth)acrylate group and one or more thiol groups with a diacrylate.

Representative reaction schemes are as follows:

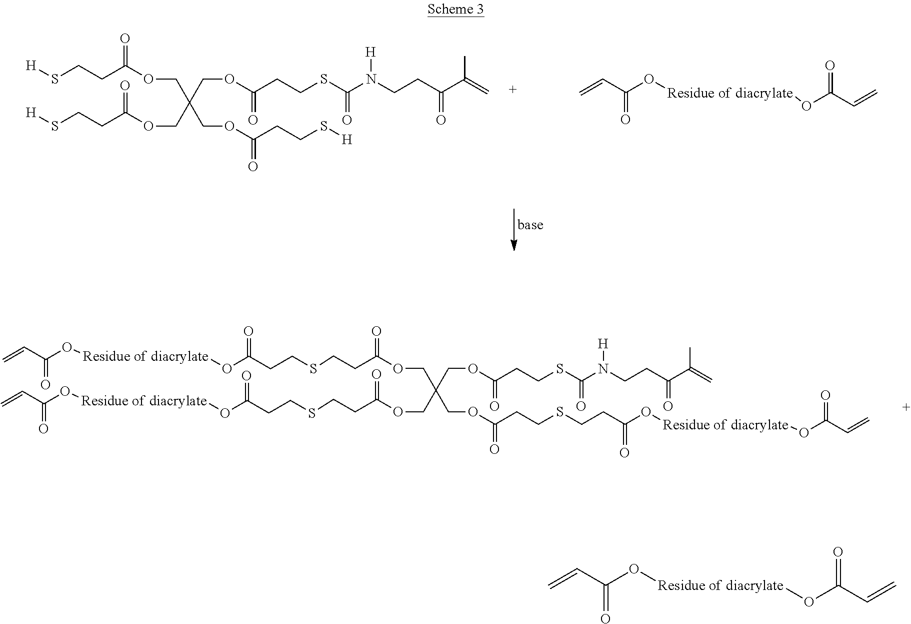

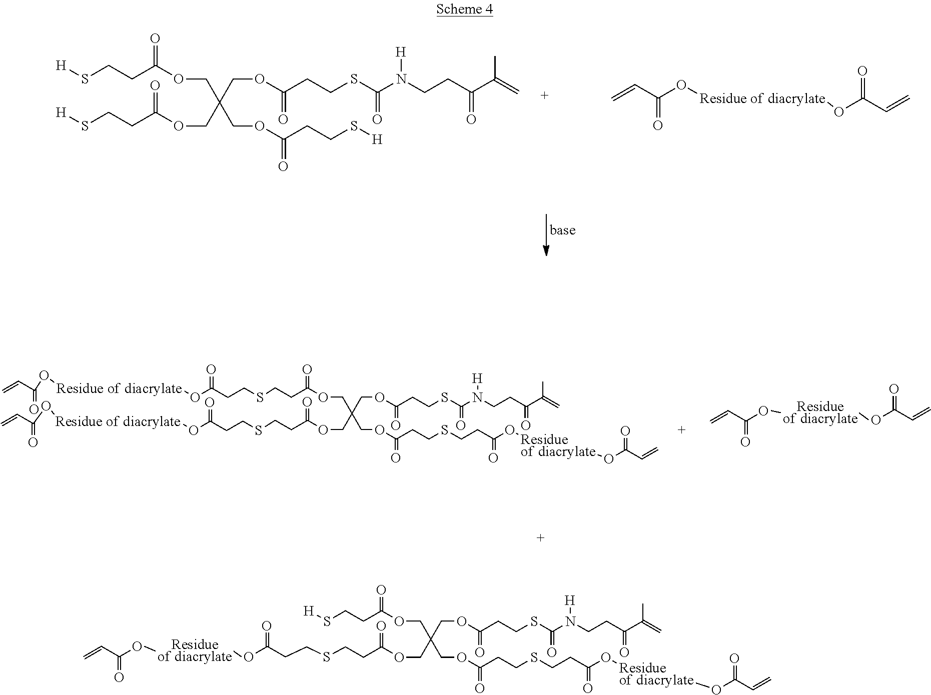

In one embodiment, the resulting compound has Formula III:

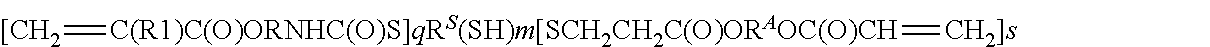

wherein

### R1 is H or CH₃,

R is a linear or branched alkylene of 2 to 10 carbons, optionally substituted with O,
R^{S} is the residue of a multithiol,
q is 1 to 5,
m is 0 to 4, (e.g. the number of unreacted thiol groups of the multithiol),
sis 1 to 5,
with the proviso that the sum of q+m+s ranges from 2 to 6 (e.g. the number of thiol groups of the multithiol).

In some embodiments, m is at least 1, 2, 3, or 4. (e.g. wherein m is 1 is depicted in Scheme 4). In some embodiments, q is 2 or 3. In some embodiments, the sum of q+m+s is 3 or 4.

Also described are polymerizable resin compositions comprising one or more (meth)acrylate monomers; and a compound comprising at least one sulfur moiety and at least one (meth)acrylate moiety, such as described herein. The polymerizable resin may comprises the same one or more (meth)acrylate monomers as utilized in the synthesis or different (meth)acrylate monomers.

The polymerizable composition may include optional additional components. Examples include organic solvent (e.g., as described hereinabove), stabilizers, colorants, photosensitizers, fillers, wetting agents, and levelling agents.

Also described is a method of making a cured composition comprising providing a (e.g. photo)polymerizable composition comprising the coupling agent as described herein: and curing the polymerizable composition. The (meth)acryl groups are subject to free-radical curing by exposure to ultraviolet radiation (UV), electron beam (e-beam), ionizing radiation (gamma rays), plasma radiation as well as thermal polymerization. In some embodiments, the method further comprises coating the polymerizable composition onto a substrate prior to curing. In some embodiments, upon radiation curing the polymerizable composition forms a film or film layer.

In some embodiments, the substrate can be a flexible substrate, for example, a web of indefinite length polymeric material. The flexible substrate or web may be stretched (e.g., along a machine direction and/or a cross direction) when moving along a web path. The flexible substrate may include, for example, polyethylene terephthalate (PET), polycarbonate (PC), polyethylene terephthalate glycol-modified (PETG), polyethylene, polyimide, polystyrene, polyurethane, etc. The processes described herein can be carried out on a roll-to-roll apparatus including one or more rollers to convey the web along the web path. The substrate may have a thickness of, for example, about 2 mm or less, about 1 mm or less, about 500 microns or less, or about 200 microns or less.

In some embodiments, radiation curing comprises exposing the (e.g. coated) composition to wavelengths of ultraviolet (UV) and visible light.

Such compositions typically include an initiator for free-radical polymerization (also known as a free-radical initiator), for example, in an effective amount). Free-radical initiators can be thermally activated (e.g., peroxides and certain azo compounds) and/or photoactivated (e.g., Norrish Type I and Type II photoinitiators). Such photoinitiators are activated by exposure to actinic radiation (e.g., ultraviolet and/or visible electromagnetic radiation).

Free-radical polymerization may be accomplished by heating or exposure to actinic radiation (e.g., ultraviolet and/or visible light, gamma rays, or an electron beam), for example, depending on the presence and/or selection of free-radical initiator(s). Of these exposure to actinic radiation is often preferred due to case of implementation.

If present, the amount of photoinitiator is typically an effective amount. In some embodiments, an effective amount of free-radical initiator comprises less than 10 percent by weight, more typically less than 7 percent by weight, and more typically less than 3 percent by weight of the total adhesive layer. It will be recognized that curing may be complete even though polymerizable (meth)acrylate groups remain.

Exemplary photoinitiators include a-cleavage photoinitiators such as benzoin and its derivatives

Examples of suitable sources of actinic radiation include actinic radiation include, for example, lasers, arc lamps (e.g., medium pressure mercury arc lamps), LED lamps, xenon flash lamps, microwave-driven lamps (e.g., equipped with H-type bulb or D-type bulb). Selection of appropriate exposure conditions will be within the capability of those skilled in the art.

The free-radically polymerizable (meth)acrylate composition can be disposed (e.g., as a continuous or discontinuous, optionally patterned film) on a surface of a substrate and then polymerized (e.g., by exposure to an effective amount of actinic radiation).

In some embodiments, the substrate is metal or comprises a surface layer of metal. In this embodiment, the free-radically polymerizable (meth)acrylate composition comprising sulfur moieties and (meth)acrylate moieties, as described herein, is applied to the metal substrate or surface layer of metal and cured, as previously described.

In this embodiment, the article comprises an organic polymer layer 7 comprising a compound comprising at least one sulfur moiety and at least one (meth)acrylate moiety. The surface of the organic polymer layer is bonded to metal 6.

In some embodiments, the metal comprises gold or silver. The metal may have a thickness of no greater than 5, 4, 3, 2, or 1 micron. In some embodiments, the metal layer have a thickness less than 1 micron, 750 nm, 500 nm, 250 nm, 100 nm, 50 nm, 25nm, or 10 nm. The thickness may be at least 10 or 25 nm. The metal layer may be continuous or discontinuous.

In some embodiments, the thickness of the organic polymer layer is at least 50 nm and no greater than 500, 400, or 300 um. In other embodiments, the thickness is at least 500 nm, 750 nm, 1 micron, 5 microns or 10 microns. In some embodiments, the thickness is no greater than 50 or 25 microns

In some embodiments, a relatively thin metal layer is bonded to a (e.g. transparent) substrate 9 with an organic polymer layer 7, such as a cured (meth)acrylate resin.

The (e.g. transparent) substrate 9 may be a sheet, plates, and films comprising any of glass, metal, organic polymer (e.g., polyethylene terephthalate (PET), polycarbonate (PC), polyethylene terephthalate glycol-modified (PETG), polyethylene, polyimide, polystyrene, or polyurethane), inorganic metal oxides, and combinations thereof.

In some embodiments, the substrate is an organic polymer film having a roughness (Ra) of less than 1 micron, 750 nm, 500 nm, or 250 nm. Thus, the interface between substrate 9 and organic polymer layer 7 (e.g. cured (meth)acrylate resin can be difficult to bond to.

The compound described herein can be used as a coupling agent for materials and methods of forming nanostructures on substrates, as described for example in WO2020/095258; incorporated herein by reference. In one embodiment, the coupling agent is present in a transfer layer comprising a cured (e.g. non-fluorinated) (meth)acrylate coating.

As described in WO2020/095258, (meth)acrylate coatings suitable for use as transfer layers may be deposited by a process of vapor coating (meth)acrylate monomer(s), optionally including added adhesion promoter(s) and/or photoinitiator(s), and cured by exposure to ultraviolet radiation (UV), electron beam (e-beam), ionizing radiation (gamma rays) or plasma radiation. This process, and suitable photocurable monomer materials for use therein, are described in U.S. Pat. No. 8,658,248 and the references incorporated therein.

Referring now to FIG. 2, an illustrative article comprising a layer of metal 6, such as silver or gold bonded to an organic polymeric layer 7, such as a cured (meth)acrylate resin. The organic layer comprises a compound comprising sulfur moieties and (meth)acrylate moieties, as described herein. The depicted layer of metal 6 comprises patterned nanostructures. The organic polymer layer 7 (e.g. cured (meth)acrylate resin) is bonded to a (e.g. transparent) substrate 9, as described above. The combination of the substrate, organic layer, and metal layer (i.e. 6, 7 and 9) may be described as a tooling film 8.

During use of the tooling film, a free-radically polymerizable (e.g. (meth)acrylate) resin 4 is applied to the metal surface of the tooling film and a substrate 2 (e.g. roughened PET) is applied to the resin 4 followed by curing of the free-radically polymerizable (e.g. (meth)acrylate) resin. When the organic polymeric layer 7 (e.g. cured (meth)acrylate resin.) comprises the compound comprising sulfur moieties and (meth)acrylate moieties, as described herein, cured resin 4 releases from metal surface of the tooling film due to the strong bond that is formed.

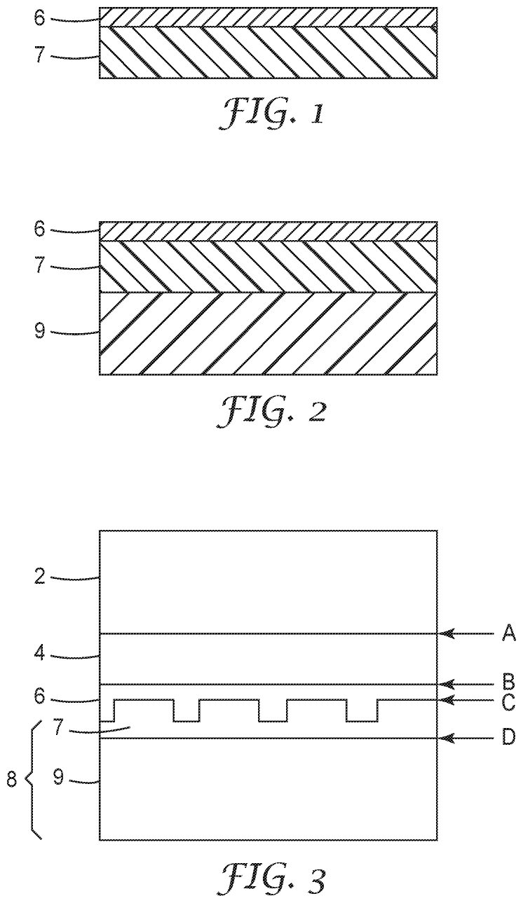

With reference to FIG. 1, in some embodiments, the amount of metal transferred (peel at interface “C” estimated by visual appearance without a microscope is at least 85, 90, 95, or 100%. When most of the metal transferred, the tooling film transparency is also high. In some embodiments, the tooling film transparency is at least 85, 90, 95, or 100%. When most of the metal transferred the transparency of the roughened PET is low. In some embodiments, the transparency of the roughened PET is less than 20, 15, 10, or 5%.

Objects and advantages of this disclosure are further illustrated by the following non-limiting examples, but the particular materials and amounts thereof recited in these examples, as well as other conditions and details, should not be construed to unduly limit this disclosure.

### EXAMPLES

Unless otherwise noted or apparent from the context, all parts, percentages, ratios, etc. in the Examples and the rest of the specification are by weight. Below table lists materials used in the examples and their sources.

| Material | |
|---|---|
| Designation | Description |
| DMAEA | 2-(Dimethylamino)ethyl acrylate, CAS number 2439-35-2, |
| | available from Sigma-Aldrich. |
| TEA | Triethylamine, available from Sigma-Aldrich. |
| NNDMAE | N,N-dimethylaminoethanol, CAS number 108-01-0, |
| | available from Sigma Aldrich |
| Hunig's base | di-isopropylethylamine, CAS number 7087-68-5, |
| | available from Sigma Aldrich. |
| Multiacrylate | A mixture of 75 parts Photomer 6210, 25 parts HDDA, |
| Mixture A | and 0.5 parts TPO. |
| (MAMA) | |
| HDDA | Hexanediol diacrylate obtained as “SR238” from Sartomer, Exton, PA. |
| | Molecular weight = 226 g/mole. |
| Photomer | Acrylated aliphatic urethane, CAS number 52404-33-8, available from IGM |
| 6210 | resins, St. Charles, IL, believed to have a molecular weight of about 950 and an |
| | acrylate equivalent weight of 475 based on NMR analysis. |
| Photomer | Phenol (4 EO) acrylate available from IGM resins, St. Charles, IL. |
| 4039 | |
| TPO | 2,4,6-trimethylbenzoyldiphenylphosphine oxide photoinitiator, |
| | CAS number 75980-60-8, obtained as “IRGACURE TPO” from BASF. |
| TEMPIC | Multithiol, as described in the written description. |
| TMMP | Multithiol, as described in the written description. |
| DMDO | Multithiol, as described in the written description. |
| GDMP | Multithiol, as described in the written description. |
| PETMP | Multithiol, as described in the written description. |
| HMDSO | Hexamethyldisiloxane, CAS number 107-46-0, available |
| | from Sigma-Aldrich. |
| Molecular | 4 angstrom, CAS number 12173-28-3, available from Sigma-Aldrich. |
| Sieves | |
| Sodium | CAS number 124-41-4, available from Sigma-Aldrich. |
| methoxide | |
| in methanol | |
| TBAH | Tetrabutylammonium hydroxide, CAS number 2052-49-5, |
| | available from Sigma-Aldrich. |
| THF | Tetrahydrofuran, available from EMD Chemicals, Billerica, NY. |
| | All THF was dried over 4 Angstrom sieves. |
| Acetic Acid | Available from EMD Chemicals, Billerica, NY |
| IEA | Isocyanatoethyl acrylate, MW 141.13, available under the |
| | trade designation “KARENZ AOI,” from Showa Denko. |
| DBTDL | Dibutyltin dilaurate, CAS number 77-58-7, available from Sigma-Aldrich. |
| 4265 | A blend of photoinitiators of CAS numbers 75980-60-8 and 7473-98-5 |
| | available as Omnirad 4265 from IGM Resins, St. Charles, Il. |
| IEM | Isocyanatoethyl methacrylate, MW 155.15, available under the trade |
| | designation “KARENZ MOI,” from Showa Denko, New York, NY. |
| IEM-EO | Isocyanatoethoxyethyl methacrylate, MW 199.2, available under the trade |
| | designation “KARENZ MOI-EG,” from Showa Denko. |

### GENERAL PROCEDURES

A nano-structured template film was prepared from a nickel template:

A nano-featured template film was prepared by die coating a mixture of PHOTOMER 6210, SR238, SR351 and IRGACURE TPO in weight ratios of 60/20/20/0.5 onto a 125 micron thick polycarbonate film with a gloss surface finish on both sides (obtained from Tekra, Inc., New Berlin, WI). The coated film was pressed against a nanostructured nickel surface attached to a steel roller controlled at 60° C. using a rubber covered roller at a speed of 15.2 m/min. The structured nickel tool was patterned with tiles of nano-scale hole features with sizes between 100 and 300 nm and 50 to 150 nm deep, interspersed with a random macroscopic roughness. The 10 random macroscopic roughness was on the order of 0.7 um Ra. The coating thickness of the resin on the film was sufficient to fully wet the nickel surface and form a rolling bead of resin as the coated film was pressed against the nanostructured nickel surface. The film was exposed to radiation from two Fusion UV lamp systems (obtained under the trade designation “F600” from Fusion UV Systems, Gaithersburg, MD) fitted with D bulbs both operating at 142 W/cm while in contact with the nanostructured nickel surface. After peeling the film from the nanostructured nickel surface, the nanostructured side of the film was exposed to radiation from a Fusion UV lamp system (“F600” from Fusion UV Systems) fitted with a D bulb operating at 142 W/cm.

The nano-structured template film was release treated:

A silicon containing release film layer [methods of forming described in U.S. Pat. Nos. 6,696,157 (David) and 8,664,323 (Iyer) and US Patent Application 2013/0229378 (Iyer)] was applied to the nanostructure template film in a parallel plate capacitively coupled plasma reactor. The chamber has a central cylindrical powered electrode with a surface area of 18.3 ft². After placing the nanostructured template film on the powered electrode, the reactor chamber was pumped down to a base pressure of less than 1.3 Pa (2 mTorr). O₂ gas was flowed into the chamber at a rate of 1000 SCCM. Treatment was carried out using a plasma enhanced CVD method by coupling RF power into the reactor at a frequency of 13.56 MHz and an applied power of 2000 watts. Treatment time was controlled by moving the nanostructured template film through the reaction zone at rate of 30 ft/min, resulting in an approximate exposure time of 10 seconds. After completing the deposition, RF power was turned off and gasses were evacuated from the reactor. Following the 1^{st} treatment, a 2^{nd} plasma treatment was carried out in the same reactor without returning the chamber to atmospheric pressure. HMDSO gas was flowed into the chamber at approximately 1750 SCCM to achieve a pressure of 9 m Torr. 13.56 MHz RF power was subsequently coupled into the reactor with an applied power of 1000 W. The nanostructured template film was then carried through the reaction zone at a rate of 30 ft/min, resulting in an approximate exposure time of 10 seconds. At the end of this treatment time, the RF power and the gas supply were stopped and the chamber was returned to atmospheric pressure.

The release treated nano-structured template film was replicated:

The (Melinex 454) PET film was pressed against the release treated nano-structured template film by laminating the films between a 90 durometer rubber roll and a steel roll controlled at 54° C. (130F). The rolls were pressed together by pressurizing two Bimba air cylinders to 0.27 MPa. The films were laminated at a speed of 3 meters/min (10 fpm). The MAMA was fed at 3-4 cc/min and produced a patterned area 10-13 cm (4-5″) wide. The film was exposed to radiation from a Fusion UV processor (Heraeus, Gaithersburg, MD) equipped with a D bulb. The PET film and cured MAMA was then separated from the release treated nano-structured template film to produce a nano-structured film.

The replicated nano-structured film was plasma treated before metallization:

An oxygen plasma was applied to the nano-structured film in a parallel plate capacitively coupled plasma reactor. The chamber has a central cylindrical powered electrode with a surface area of 1.7 m² (18.3 ft²). After placing the nano-structured film on the powered electrode, the reactor chamber was pumped down to a base pressure of less than 0.5 Pa (0.8 m Torr). O₂ gas was flowed into the chamber at a rate of 1000 SCCM (standard cubic centimeters per minute). Treatment was carried out using a plasma enhanced CVD method by coupling RF power into the reactor at a frequency of 13.56 MHz and an applied power of 6000 watts. Treatment time was controlled by moving the nano-structured film through the reaction zone at rate of 10.6 meter/min (35 ft/min) resulting in an approximate exposure time of 8 seconds. After completing the deposition, RF power was turned off and gasses were evacuated from the reactor, and the chamber was returned to atmospheric pressure.

Metal was deposited on the plasma treated replicated nano-structured film:

The plasma treated nano-structured films were then deposited with a metal layer to arrive at the final nano-structured metallized film. The nanohole structures were vapor coated with Ag/Au using a roll-to-roll dc sputtering system with the target parallel to the substrate film during deposition. The sputtering target was 85% Ag/15% Au with dimensions 9.8 cm×53.3 cm×0.64 cm. The Ag/Au was deposited at an Argon pressure of 0.4 Pa and a power of 3.8 kW to thicknesses of either 50, 100, or 250 nm by varying the line speed and the number of passes through the deposition zone. For 50 nm thickness the line speed was 2.67 m/min, for 100 nm thickness the line speed was 1.34 m/min, and for 250 nm thickness the film made two passes through the deposition zone each at 1.07 m/min. Following the sputtering treatment, the pressure was returned to ambient pressure and the metallized structured films were removed from the machine.

### Roughened PET:

A PET (Melinex ST504) film substrate (Tekra, Inc.) was roughened by depositing a discontinuous silicon containing layer using PECVD while either simultaneously or sequentially etching the surface with a reactive species as described in U.S. Pat. No. 10,134,566 and U.S. Pat. No. 8,634,146. Reactive ion etching was carried out on PET film substrate in the same home-built reactor chamber used to deposit the release layer. After placing the film on the powered electrode, the reactor chamber was pumped down to a base pressure of less than 1.3 Pa (1 m Torr). HMDSO and O₂ gases were flowed into the chamber at rates of 18 and 750 SCCM, respectively. 13.56 MHz RF power was subsequently coupled into the reactor with an applied power of 7500 W. The film was then carried through the reaction zone at a rate of 15 ft/min, to achieve an exposure time of approximately 20 sec. At the end of this treatment time, the RF power and the gas supply were stopped and the chamber was returned to atmospheric pressure.

### Resin Adhesion Test:

Multithiol and MAMA were combined in at the levels indicated in the examples using roughly 10 g batches in a 20 cc cup. The components were mixed in a speed-mixer at 3000 rpm for 30 seconds. The polymerizable resin mixture was then left at room temperature for a measured amount of time. The mixture was then coated onto the final nano-structured metallized film using a #40 Meyer rod. The film was then placed in an Athena Blue oven (model M01420A), set to 70 degrees Celsius, for two minutes. The film was hand-laminated to the roughened PET and cured for 30 s with a UV-LED unit has an array of 16×4 385 nm UV-LEDs arranged in a 16 cm by 4 cm area, and is powered at 2 amps at 32 volts resulting in 28 mW/cm². The film was placed onto a table with the mold on the bottom and the roughened PET on top; the roughened PET was peeled from the tooling film at a peel rate of about 5 cm/sec.

Multithiol and isocyanato(meth)acrylate compounds were also made, the details of which are described below. The polymerizable resin was coated onto the final nano-structured metallized film, laminated to the roughened PET and, cured in the same manner just described.

FIG. 1 depicts the interfaces of the assembled layers. The adhesion between the resin and the roughened PET (interface “A”) in FIG. 1 was typically quite good. The adhesion between the layers within the tooling film (interface “D”) were also typically quite good. The Au/Ag layer either released from the tooling film (interface “C”) or did not stick to the resin (interface “B”). The amount of metal transferred (peel at interface “C”) was estimated by eye. If all of the metal was transferred, the roughened PET was reflective and not transparent, and the tooling film was transparent. The transparency of the tooling film and the roughened PET were measured on a haze-gard (4775 “haze-gard i” BYK instruments). Samples were oriented on the haze-gard with light first passing through the PET and roughened PET respectively. The measured transparency of the tooling film and roughened PET are quantitative ways to infer the adhesion strength of the resin/metal layer compared to the metal/tooling film layer.

The following Comparative Examples (CEs) show that unreacted multithiols and multiacrylates (without a base) are ineffective as silver-gold coupling agents. Also, a control sample of a multiacrylate resin without coupling agent was ineffective. The thickness of the Ag/Au layer was 100 nm.

TABLE 1

| | Multi- | | | Rough- | | | Rough- |
|---|---|---|---|---|---|---|---|
| | thiol 5 | Peel % | Tooling | ened | Peel % | Tooling | ened |
| | parts in | at Inter- | film | PET | at Inter- | film | PET |
| | MAMA | face C | Trans at | Trans at | face C | Trans at | Trans at |
| Ex. | Resin | 2 h | 2 h | 2 h | 2 days | 2 days | 2 days |
| CE-1 | None | 10 | NA | NA | NA | NA | NA |
| | (100% | | | | | | |
| | MAMA | | | | | | |
| | Resin) | | | | | | |
| CE-2 | TMMP | 0 | 0.18 | 91.5 | 0 | 0.2 | 93.3 |
| CE-3 | PETMP | 0 | 0.16 | 91.7 | 0 | 0.2 | 92.9 |
| CE-4 | TEMPIC | 0 | 0.18 | 91.2 | 0 | 0.2 | 91.6 |
| CE-5 | GDMP | 0 | 0.11 | 92.4 | 0 | 0.2 | 91.4 |
| CE-6 | PEI | 0 | 0.18 | 91.3 | 0 | 0.3 | 91.3 |

The following examples consist of 95 parts of MAMA. 5 parts of the indicated multithiol. and 0.5 parts solids of Hunig's base added as a 10% solids solution in THF. The solutions were stored for the times indicated and evaluated (coated. cured. and peel tested). The thickness of the Ag/Au layer was 50 nm.

TABLE 2

| | | Storage | | | |
|---|---|---|---|---|---|
| | | Time of | Peel % at | Tooling film | Roughened |
| | | Solution | Interface C at | Trans at that | PET Trans at |
| Ex. | Multithiol | Hours | that time | time | that time |
| 1 | TMMP | 2.5 | 20 | 22 | 82 |
| 2 | TMMP | 4.16 | 100 | 91 | 9 |
| 3 | TMMP | 6 | 100 | 86 | 11 |
| 4 | TMMP | 24 | 100 | 91 | 5 |
| 5 | PETMP | 2.5 | 100 | 92 | 5 |
| 6 | PETMP | 4.16 | 100 | 92 | 4 |
| 7 | PETMP | 6 | 100 | 89 | 17 |
| 8 | PETMP | 24 | 100 | 87 | 10 |
| 9 | GDMP | 2.5 | 0 | 4 | 91 |
| 10 | GDMP | 4.16 | 10 | 20 | 80 |
| 11 | GDMP | 6 | 30 | 61 | 50 |
| 12 | GDMP | 24 | 100 | 92 | 4 |
| 13 | TEMPIC | 2.5 | 35 | 36 | 68 |
| 14 | TEMPIC | 4.16 | 100 | 91 | 5 |
| 15 | TEMPIC | 6 | 100 | 94 | 5 |
| 16 | TEMPIC | 24 | 100 | 91 | 5 |
| 17 | PEI | 2.5 | 0 | 4 | 92 |
| 18 | PEI | 4.16 | 0 | 6 | 91 |
| 19 | PEI | 6 | 0 | 6 | 91 |
| 20 | PEI | 24 | 100 | 92 | 5 |
| 21 | DMDO | 2.5 | 0 | 4 | 91 |
| 22 | DMDO | 4.16 | 0 | 4 | 92 |
| 23 | DMDO | 6 | 0 | 4 | 92 |
| 24 | DMDO | 24 | 90 | 82 | 25 |

During storage the multithiol reacted with the MAMA. This reaction was confirmed with ¹³C, ¹H, and HSQC (Heteronuclear Single Quantum Coherence) proton-carbon correlation Nuclear Magnetic Resonance analysis. At time zero and other times when the Peel % at Interface C was zero, the solution contained two peaks corresponding to the unreacted multithiol and unreacted MAMA. At reaction times when the Peel % at Interface C was 100%, the peak corresponding to the unreacted multithiol was much smaller and new peaks were present corresponding to the reaction product of the multithiol with MAMA. At reaction times when the Peel % at Interface C was greater than zero and less than 100%, peaks corresponding to both the multithiol and it's reaction product with MAMA were present and the reaction product peak was smaller than at reaction times wherein the Peel % at Interface C was 100%.

Additional bases were evaluated formulations containing 95 parts of MAMA, 5 parts of PETMP. The bases were added as a 10% solids solution in THF. The solutions were allowed to react for the times indicated and evaluated (coated, cured, and peel tested). The thickness of the Ag/Au layer was 50 nm. Example CE-7 control was 100% MAMA, without coupling agent.

TABLE 3

| | | | | Peel % | | Rough- |
|---|---|---|---|---|---|---|
| | | | Reaction | at Inter- | Tooling | ened |
| | | Solids | Time of | face C | film | PET |
| | | parts | Solution | at that | Trans at | Trans at |
| Ex. | Base | base | in hours | time | that time | that time |
| 25 | TEA | 0.5 | 2.7 | 90 | 91 | 5 |
| 26 | TEA | 0.5 | 4.3 | 95 | 89 | 7 |
| 27 | TEA | 0.5 | 6 | 100 | 91 | 6 |
| 28 | TEA | 0.5 | 24 | 100 | 90 | 8 |
| 29 | NNDMAE | 0.5 | 2.7 | 100 | 91 | 4 |
| 30 | NNDMAE | 0.5 | 4.3 | 100 | 91 | 6 |
| 31 | NNDMAE | 0.5 | 6 | 100 | 91 | 5 |
| 32 | NNDMAE | 0.5 | 24 | 100 | 89 | 7 |
| 33 | Hunig's | 0.5 | 2.7 | 100 | 92 | 5 |
| 34 | Hunig's | 0.5 | 4.3 | 100 | 92 | 4 |
| 35 | Hunig's | 0.5 | 6 | 100 | 89 | 17 |
| 36 | Hunig's | 0.5 | 24 | 100 | 87 | 10 |
| CE-7 | NA | NA | 0 | 2 | — | — |

The acrylate equivalent weight of Photomer 6210 is 475 g/eq. The acrylate equivalent weight of HDDA is 226/2=113 g/eq. A 75:25 mixture of Photomer 6210: HIDDA has a weighted average acrylate equivalent weight of 0.75*475+0.25*113 or 384.53. Assuming 100 g of total resin, the equivalents and equivalent ratio can be calculated as follows:

TABLE 4A

| | Parts | | Parts | Equivalents | Ratio of |
|---|---|---|---|---|---|
| | PETMP | | MAMA | of | Equivalents |
| | (126.5 | Equivalents | (Acrylate | acrylates | of |
| Example | thiol EW) | of Thiol | EW 384.53) | in MAMA | Acrylate:Thiol |
| 37 | 1 | 0.00791 | 99 | 0.257455 | 32.56 |
| 38 | 2.5 | 0.019763 | 97.5 | 0.253554 | 12.89 |
| 39 | 5 | 0.03953 | 95 | 0.247052 | 6.25 |
| 40 | 7.5 | 0.059289 | 92.5 | 0.2400551 | 4.05 |
| 41 | 10 | 0.07905 | 90 | 0.23405 | 2.96 |
| 42 | 15 | 0.118577 | 85 | 0.221047 | 1.86 |
| 43 | 20 | 0.158103 | 80 | 0.208044 | 1.32 |

The following examples used MAMA and PETMP totaling 100 parts, with addition of 0.5 parts of Hunig's base (delivered as a 10% solution in THF). The amount PETMP used is given. For example, if PETMP is 1 part, MAMA is 99 parts, and if PETMP is 20 parts then MAMA is 80 parts. At 2 h of aging, the coating is coated and cured, and the results are reported below. The thickness of the Ag/Au layer was 100 nm.

TABLE 4B

| | Ratio of | | Peel % at | Tooling | Roughened |
|---|---|---|---|---|---|
| | Equivalents | | Inter- | film | PET Trans |
| | of Acry- | Parts | face C | Trans | at Time |
| Example | late:Thiol | PETMP | 2 h | at 2 h | 2 h |
| 37 | 32.56 | 1 | 100 | 92 | 1.4 |
| 38 | 12.89 | 2.5 | 100 | 91 | 0.5 |
| 39 | 6.25 | 5 | 100 | 91 | 0.95 |
| 40 | 4.05 | 7.5 | 100 | 92 | 0.2 |
| 41 | 2.96 | 10 | 100 | 94 | 1.1 |
| 42 | 1.86 | 15 | Peels at | | |
| | | | inter- | | |
| | | | face D | | |
| 43 | 1.32 | 20 | Peels at | | |
| | | | inter- | | |
| | | | face D | | |

The Table below shows Examples 44-47 in which no solvent is added to compositions. The MAMA and multithiol are spin mixed at 3000 rpm for 5 min before the base is added, and the resulting composition is spin mixed at 3000 rpm for 1 min. In the case of DMAEA, the base is acrylate functional. Also shown are Comparative Examples run concurrently.

TABLE 5

| | | Peel % at Interface C | | | Tooling film transparency | | | Roughened PET Trans | | |
|---|---|---|---|---|---|---|---|---|---|---|
| Ex. | Composition | at given time | | | at given time | | | at given time | | |
| | Dwell time | 30 min | 2.5 h | 24 h | 30 min | 2.5 h | 24 h | 30 min | 2.5 h | 24 h |
| | since mixing | | | | | | | | | |
| 44 | 9.5 g MAMA, | 0 | 0 | 100 | 0.03 | 0.3 | 93.2 | 92 | 91 | 1.9 |
| | 0.5 g PETMP, | | | | | | | | | |
| | 0.05 g DMAEA | | | | | | | | | |
| 45 | 9.5 g MAMA, | 0 | 0 | 100 | 0.03 | 0.5 | 93.3 | 92 | 91 | 0.71 |
| | 0.5 g PETMP, | | | | | | | | | |
| | 0.1 g DMAEA | | | | | | | | | |
| 46 | 9.5 g MAMA, | 60 | 100 | 100 | 73 | 93 | 93 | 21 | 0.1 | 0.93 |
| | 0.5 g PETMP, | | | | | | | | | |
| | 0.05 g Hunig's | | | | | | | | | |
| | base | | | | | | | | | |
| 47 | 9.5 g MAMA, | 40 | 100 | 100 | 32 | 92 | 93.1 | 61 | 0.1 | 0.98 |
| | 0.5 g PETMP, | | | | | | | | | |
| | 0.1 g Hunigs's | | | | | | | | | |
| | base | | | | | | | | | |
| | Time laminated in | 2 min | 4 min | 6 min | 2 min | 4 min | 6 min | 2 min | 4 min | 6 min |
| | oven before peel | | | | | | | | | |
| CE-8 | MAMA only | 0 | 0 | 0 | 0.1 | 0.05 | 0.1 | 91 | 91 | 90 |
| | Dwell time | | 2 h | 24 h | | 2 h | 24 h | | 2 h | 24 h |
| | since mixing | | | | | | | | | |
| CE-9 | 9.5 MAMA, | | 0 | 0 | | 0.3 | 0.07 | | 92 | 92.3 |
| | 0.5 PETMP | | | | | | | | | |
| | (no base) | | | | | | | | | |
| CE-10 | 9.5 MAMA, 1.25 g | | 40 | 5 | | 75 | 16.1 | | 31 | 89.6 |
| | of 40% Hunig's | | | | | | | | | |
| | base in THF | | | | | | | | | |

The following examples used 95 parts of the indicated diacrylate and 5 parts PETMP, totaling 100 parts, with addition of 0.5 parts of Hunig's base (delivered as a 10% solution in THF). Photomer 6210 and Photomer 4039 each included 0.5 parts of 4265. Comparative examples with Photomer 6210 and Photomer 4039 had only the diacrylate, 0.5 parts of 4265 with no base and no PETMP. The thickness of the Ag/Au layer was 250 nm.

TABLE 6

| | 95 parts of | Peel % at | Peel % at |
|---|---|---|---|
| | following | Interface | Interface |
| Ex. | diacrylate | C 2 h | C 4 h |
| 48 | Photomer 6210 | 60 | 100 |
| 49 | Photomer 4039 | 15 | 90 |
| 50 | MAPA | 10 | 100 |
| CE-11 | Photomer 6210 | 0 | |
| CE-12 | Photomer 4039 | 0 | |
| CE-13 | MAPA | 0 | |

Reaction of Multithiol with Isocyanato(meth)acrylates

All reactions were adjusted to 40% solids with respect to the multithiol and isocyanato (meth)acrylate. In the example PE-1 below there are 17.18 g solids which are diluted to a total weight of 42.96 g with 25.78 g solvent. The 10% solutions of Hunig's base and acetic acid are considered part of the solvent added (0.63+0.58+24.57=25.78). The multithiol was always diluted in an equal weight of THF. The isocyanatoalkyl (meth)acrylate was diluted with the remainder of the solvent before addition to the multithiol (25.78−(0.63+0.58+12.5)=12.07 g). All THF was dried over 4 Angstrom sieves.

### Preparation 1 (PE-1) TMMP:IEM=3:1

To a solution of TMMP 12.50 g (0.0905 eq, 138 thiol EW) in 12.50 g THF and 0.625 g of a 10% solution of Hunig's base (0.000483 eq) cooled to −11.7° C. under dry air was added via a pressure equalizing dropping funnel 4.68 g (0.0302 eq) of IEM in 12.07 g THF over 1 h. At that time an aliquot was removed for FTIR analysis and no NCO was found at 2265 cm⁻¹. The reaction was treated with 0.581 g of a 10% solution of acetic acid (0.000967 eq) in THF. The reaction was bottled and stored in a −30° C. freezer. An aliquot of the reaction was diluted with an equal volume of CDCl₃ and found to be consistent with the desired structure by ¹H NMR.

TABLE 7

| | | | | | | | | | % Wt of | |
|---|---|---|---|---|---|---|---|---|---|---|
| | | | Isocyanato- | | | 10% in THF | 10% in | | isocy- | Wt % |
| | | | (meth) | | | Hunig's | THF Acetic | THF | anate | thiol |
| | Multithiol | | acrylate | | SH:NCO | base | Acid | Added | in | in |
| Ex. | Type | Amt (g) | Type | Amt (g) | ratio | (g) | (g) | (g) | reaction | reaction) |
| PE-1 | TMMP | 12.50 | IEM | 4.68 | 3:1 | 0.63 | 0.58 | 24.57 | 27.2 | 72.8 |
| PE-2 | TMMP | 12.50 | IEM-EO | 6.01 | 3:1 | 0.63 | 0.58 | 26.57 | 32.5 | 67.5 |
| PE-3 | PETMP | 12.50 | IEM | 3.83 | 3:1 | 0.63 | 0.58 | 23.29 | 23.5 | 76.5 |
| PE-4 | PETMP | 12.50 | IEM-EO | 2.46 | 8:1 | 0.63 | 0.58 | 21.23 | 16.4 | 83.6 |
| PE-5 | PETMP | 12.50 | IEM-EO | 4.92 | 4:1 | 0.63 | 0.58 | 24.82 | 28.2 | 71.8 |
| PE-6 | PETMP | 12.50 | IEM-EO | 7.38 | 2.66:1 | 0.63 | 0.58 | 28.62 | 37.1 | 62.9 |
| PE-7 | PETMP | 10.00 | IEM-EO | 7.87 | 2:1 | 0.50 | 0.47 | 25.85 | 44.0 | 56.0 |
| PE-8 | PETMP | 8.00 | IEM-EO | 9.45 | 1.66:1 | 0.40 | 0.37 | 25.40 | 54.2 | 45.8 |
| PE-9 | GDMP | 12.5 | IEM | 3.93 | 4:1 | 0.63 | 0.58 | 23.43 | 23.9 | 76.1 |
| PE-10 | GDMP | 12.5 | IEM-EO | 5.04 | 4:1 | 0.63 | 0.58 | 25.10 | 28.7 | 71.3 |
| PE-11 | TEMPIC | 12.5 | IEM | 3.55 | 3:1 | 0.63 | 0.58 | 22.87 | 22.1 | 77.9 |
| PE-12 | TEMPIC | 12.5 | IEM-EO | 4.56 | 3:1 | 0.63 | 0.58 | 24.38 | 26.7 | 73.3 |
| PE-13 | PEI | 12.5 | IEM-EO | 4.61 | 4:1 | 0.63 | 0.58 | 24.46 | 26.9 | 73.1 |
| PE-14 | PETMP | 12.50 | IEM-EO | 4.92 | 4:1 | 0.63 | 0.58 | 24.82 | 28.2 | 71.8 |
| PE-15 | TMMP | 12.50 | IEM-EO | 18.04 | 1:1 | 0.63 | 0.58 | 44.61 | 59.1 | 40.9 |
| PE-16 | PETMP | 12.5 | IEM-EO | 7.38 | 2.66:1 | 0.63* | 0.58 | 28.61 | 37.1 | 62.9 |
| PE-17** | TMMP | 12.50 | IEA | 4.26 | 3:1 | 0.63 | 0.58 | 40.69 | 25.4 | 74.6 |

PE-14 was a repeat of PE-5
*10% Triethylamine in THF is the base used
**Addition of the IEA to multithiol performed at −25 to −30° C.

The following examples consist of 95 parts of MAMA, and 5 parts of the indicated PE. The solutions were aged for the times indicated and evaluated (coated, cured, and peel tested) as the solutions aged. The thickness of the Ag/Au layer was 100 nm.

TABLE 8

| | | | | Rough- | | | Rough- |
|---|---|---|---|---|---|---|---|
| | | Peel % | Tooling | ened | Peel % | Tooling | ened |
| | | at Inter- | film | PET | at Inter- | film | PET |
| | Prepa- | face C | Trans | Trans at | face C | Trans at | Trans at |
| Ex. | ration | 2 h | at 2 h | Time 2 h | 2 days | 2 days | 2 days |
| 51 | PE-1 | 10 | 14.4 | 85.2 | 50 | 85.3 | 13 |
| 52 | PE-2 | 30 | 33.1 | 78 | 95 | 90 | 1.8 |
| 53 | PE-3 | 10 | 8.8 | 88.8 | 100 | 91.5 | 1.2 |
| 54 | PE-4 | 5 | 3.6 | 89.9 | 100 | 91.8 | 0.4 |
| 55 | PE-5 | 50 | 78.4 | 28.3 | 100 | 91.6 | 0.9 |
| 56 | PE-6 | 100 | 91.9 | 0.44 | 100 | 91.6 | 0.6 |
| 57 | PE-7 | 100 | 91.4 | 0.98 | 100 | 91.9 | 0.5 |
| 58 | PE-8 | 100 | 91.2 | 0.45 | 100 | 91.5 | 0.8 |
| 59 | PE-9 | 0 | 0.35 | 91 | 0 | 1.3 | 91.3 |
| 60 | PE-10 | 0 | 0.53 | 91.1 | 0 | 3.4 | 93.5 |
| 61 | PE-11 | 10 | 7.6 | 89.2 | 100 | 90.7 | 9.4 |
| 62 | PE-12 | 30 | 60.5 | 54 | 100 | 90.3 | 0.9 |
| 63 | PE-13 | 50 | 66.6 | 35.9 | 60 | 84.3 | 13.7 |

Dithiols and secondary thiols can be less preferred. However, higher concentration of multithiol isocyanato acrylate adducts could provide greater % peel at interface C.

The following examples used MAMA and PE-5 totaling 100 parts. The amount PE-5 used is given. For example, if PE-5 is 1 part, MAMA is 99 parts, and if PE-5 is 20 parts then MAMA is 80 parts. The solutions were aged for the times indicated and evaluated (coated, cured, and peel tested) as the solutions aged. The thickness of the Ag/Au layer was 100 nm. At 2 days of aging, the coating when coated and cured, peels the silver-gold from the roughened PET.

TABLE 9

| | | | | Rough- | | | Rough- |
|---|---|---|---|---|---|---|---|
| | | Peel % | Tooling | ened | Peel % | Tooling | ened |
| | | at Inter- | film | PET | at Inter- | film | PET |
| | Parts | face | Trans at | Trans at | face C | Trans at | Trans at |
| Ex. | PE-5 | C 2 h | 2 h | 2 h | 2 days | 2 days | 2 days |
| 64 | 1 | 60 | 79.9 | 18.7 | 100 | 92 | 0.43 |
| 65 | 2.5 | 60 | 83.4 | 14.9 | 100 | 91.3 | 1.5 |
| 66 | 5 | 70 | 90 | 3.7 | 100 | 91.8 | 0.59 |
| 67 | 7.5 | 75 | 89.9 | 4.2 | 100 | 92 | 0.45 |
| 68 | 10 | 75 | 90.1 | 4.5 | 100 | 92 | 0.3 |
| 69 | 20 | 80 | 85.5 | 12.6 | 100 | 92 | 0.61 |

The following examples consist of 95 parts of MAMA, and 5 parts of the indicated PE or the Composition indicated. The solutions were aged for the times indicated and then evaluated (coated, cured, and peel tested) as the solutions aged. The thickness of the Ag/Au layer was 250 10 nm.

TABLE 10

| Exam- | Preparation or | Peel % at Interface C | | | Tooling film transparency | | | Roughened PET Trans | | |
|---|---|---|---|---|---|---|---|---|---|---|
| ple | Composition | at given time | | | at given time | | | at given time | | |
| | | 30 min | 2.5 h | 24 h | 30 min | 2.5 h | 24 h | 30 min | 2.5 h | 24 h |
| 70 | PE-15 | 100 | 100 | 100 | 91 | 91.5 | 93 | 0.5 | 1 | 1.7 |
| 71 | PE-16 | 20 | 100 | 100 | 71 | 91.5 | 93.4 | 70 | 0.6 | 0.17 |
| | | 30 min | 4 h | 24 h | 30 min | 4 h | 24 h | 30 min | 4 h | 24 h |
| 72 | 9.5 g 6210, | 95 | 100 | 100 | 89 | 90 | 93.3 | 3 | 2 | 0.52 |
| | 0.1 g 4265 | | | | | | | | | |
| | 1.25 g PE-16 | | | | | | | | | |
| | (40% solids) | | | | | | | | | |
| | | 1 h | 3 h | 24 h | 1 h | 3 h | 24 h | 1 h | 3 h | 24 h |
| 73 | PE-17 | 0 | 20 | | 9.8 | 38.9 | | 87.5 | 80 | |
| | | 2 h | 4 h | 6 h | 2 h | 4 h | 6 h | 2 h | 4 h | 6 h |
| CE | MAMA only | 0 | 0 | 0 | 0.1 | 0.05 | 0.1 | 91 | 91 | 90 |

The following experiments were run with the 95 parts of MAMA and 5 parts PE-15. The same sample of coating solution was evaluated (coated, cured, and peel tested) as the sample aged at 0, 2, 4, 6, 8, and 28 h, while at the same time coating (diluted in an equal volume of CDCl₃) was evaluated by NMR at as close as possible to the same time intervals. The thickness of the Ag/Au layer was 100 nm. The Peel % increases over time, and ¹H NMR, shows Michael adduct peaks from 2.83-2.78 ppm and 2.67-2.64 ppm. The peaks increase as the time increases.

TABLE 11

| | | Peel % at | Tooling | Roughened |
|---|---|---|---|---|
| | Time | Inter- | film | PET Trans |
| Example | (h) | face C | Trans at | at Time A |
| 74 | 0 | — | — | — |
| 75 | 2 | 15 | 32 | 60 |
| 76 | 4 | 15 | 78 | 16 |
| 77 | 6 | 50 | 75 | 34 |
| 78 | 8 | 80 | 84 | 15.6 |
| 79 | 28 | 100 | 91 | 1.1 |

Additional bases were evaluated in formulations containing 95 parts of MAMA and 5 parts of PE-15. The bases were added as a 10% solids solution in THF. The solutions were allowed to react for the times indicated and evaluated (coated, cured, and peel tested). The thickness of the Ag/Au layer was 50 nm. Example CE-7 control was 100% MAMA without coupling agent.

TABLE 12

| | | | | | | Rough- |
|---|---|---|---|---|---|---|
| | | | | Peel % | Tooling | ened |
| | | | Reaction | at Inter- | film | PET |
| | | Solids | Time of | face C | Trans | Trans |
| Exam- | | parts | Solution | at that | at that | at that |
| ple | Base | base | in h | time | time | time |
| 80 | TEA | 0.5 | 2.5 | 100 | 92 | 5 |
| 81 | TEA | 0.5 | 6 | 100 | 91 | 6 |
| 82 | NNDMAE | | 2.6 | 100 | 91 | 4 |
| 83 | NNDMAE | 0.5 | 6 | 100 | 91 | 5 |

Hunig's base was tested at the solids parts and age of solution and noted for their effectiveness in improving Peel % in formulations containing 95 parts of MAMA, 5 parts of PETMP. The Hunig's base were added as a 10% solids solution in THF, except as noted. The solutions were aged for the times indicated and evaluated (coated, cured, and peel tested) as the solutions aged. The thickness of the Ag/Au layer was 100 nm.

| | | | | | | Rough- |
|---|---|---|---|---|---|---|
| | | | | Peel % | Tooling | ened |
| | | | | at Inter- | film | PET |
| | | Solids | Age of | face C | Trans | Trans |
| Exam- | | parts | Solution | At that | at that | at that |
| ple | Base | base | in h | time | time | time |
| 84 | Hunig's | 0.025 | 2 | 0 | 0.2 | 91 |
| 85 | Hunig's | 0.025 | 26 | 0 | 1.6 | 90.3 |
| 86 | Hunig's | 0.25 | 2 | 60 | 83.8 | 8.8 |
| 87 | Hunig's | 0.25 | 26 | 100 | 92 | 0.8 |
| 88 | Hunig's | 2.5 | 2 | 100 | 92 | 0.7 |
| 89 | Hunig's | 2.5 | 26 | 50 | 45.4 | 28 |
| 90 | Hunig's | 0.1 | 2 | 0 | 0.2 | 91.4 |
| 91 | Hunig's | 0.1 | 4 | 0 | 3.3 | 91 |
| 92 | Hunig's | 0.1 | 6 | 40 | 36 | 71 |
| 93 | Hunig's | 0.1 | 8 | 50 | 72 | 29 |
| 94 | Hunig's | 0.1 | 28 | 100 | 91 | 2.4 |
| 95 | Hunig's- | 0.5 | 2 h | 100 | 91.6 | 1.8 |
| | no THF | | | | | |

5. An article comprising:
an organic polymer layer comprising a compound comprising at least one sulfur moiety and at least one (meth)acrylate moiety according to claim 1; wherein a surface of the organic polymer layer is bonded to metal.

6. The article of claim 5 wherein the metal comprises gold or silver.

7. The article of claim 5 wherein the metal has a thickness of no greater than 5 microns, 1 micron, 500 nm, 250 nm, 100 nm, or 50 nm.

8. The article of claim 5 wherein the metal is a continuous layer.

9. The article of claim 5 wherein the opposing surface of the metal has microstructured or nanostructured surface.

10. The article of claim 5 wherein the organic polymer layer comprises cured multi (meth)acrylate monomers and less than 15 wt. % of moieties of a multithiol.

11. The article of claim 5 wherein the opposing surface of the organic polymer layer is disposed on a substrate.

12. The article of claim 11 wherein the surface of the organic polymer layer has a surface roughness of less than 1 micron, 750 nm, 500 nm, or 250 nm.

13. The article of claim 11 wherein the substrate is a film that is transparent.

10-12. (cancelled)

1. A compound of 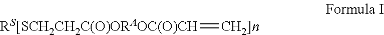
wherein
R^{S} is the residue of a multithiol,
R^{A} is independently a residue of a diacrylate, and
n is 2 to 6; 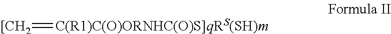
wherein
R1 is H or CH₃,
R is a linear or branched alkylene of 2 to 10 carbons, optionally substituted with O,
R^{S} is the residue of a multithiol,
q is 1 to 5,
m is 1 to 5,
with the proviso that the sum of q+m ranges from 2 to 6;
or 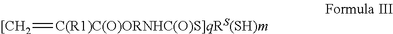
wherein
R1 is H or CH₃,
R is a linear or branched alkylene of 2 to 10 carbons, optionally substituted with O,
RS is the residue of a multithiol,
q is 1 to 5,
m is 1 to 5,
with the proviso that the sum of q+m ranges from 2 to 6.

14-17. (cancelled)

2. The compound of claim 1 wherein m of Formula III is at least 1.

3. A polymerizable resin composition comprising:
one or more (meth)acrylate monomers; and
at least one compound comprising at least one sulfur moiety and at least one (meth)acrylate moiety according to claim 1.

4. The polymerizable resin composition of claim 3 wherein the composition is cured.

21-24. (cancelled)
